(19) United States
(12) Patent Application Publication
(10) Pub. No.: US 2019/0265459 A1
(43) Pub. Date: Aug. 29, 2019

(54) TEMPERATURE MEASUREMENT SYSTEM AND ENDOSCOPE

(71) Applicant: OLYMPUS CORPORATION, Tokyo (JP)

(72) Inventors: Yasuo HIRATA, Tokyo (JP); Susumu TAKAHASHI, Iruma-shi (JP)

(73) Assignee: OLYMPUS CORPORATION, Tokyo (JP)

(21) Appl. No.: 16/281,195

(22) Filed: Feb. 21, 2019

(30) Foreign Application Priority Data
Feb. 23, 2018 (JP) 2018-030894

(51) Int. Cl. G02B 23/26; G02B 23/24; A61B 1/00; A61B 1/005

(52) U.S. Cl. CPC G02B 23/26; G02B 23/2461; A61B 1/05; A61B 1/005; A61B 1/00165

(57) ABSTRACT

An endoscope system includes an observation optical system, a beam splitter configured to branch light from the observation optical system into first light including visible light from a subject and second light for temperature measurement, an image pickup device configure to receive the first light from the beam splitter and pick up an image of the subject, a spectroscope configured to receive the second light from the beam splitter and measure plural spectral radiances of the subject, and a personal computer configured to calculate the temperature of the subject based on an output signal including the plural spectral radiances of the spectroscope.

### CROSS REFERENCE TO RELATED APPLICATION

This application claims benefit of Japanese Application No. 2018-030894 filed in Japan on Feb. 23, 2018, the contents of which are incorporated by this reference.

### BACKGROUND OF THE INVENTION

### 1. Field of the Invention

The present invention relates to a temperature measurement system and an endoscope, and particularly to a temperature measurement system and an endoscope that are capable of acquiring an image of a subject and measuring the temperature of the subject.

### 2. Description of the Related Art

Conventionally, a radiation thermometer for measuring the temperature of a subject has been widely used. For example, Japanese Patent Application Laid-Open Publication No. H7-260581 discloses a technique in which a spectral radiance of an electromagnetic wave radiated from a subject under a high temperature environment is measured with respect to plural different wavelengths by a radiation thermometer, and the temperature is measured from two different wavelengths out of the plural spectral radiances.

An endoscope for observing the inside of a subject is also widely used. For example, Japanese Patent Application Laid-Open Publication No. 2010-249944 discloses an endoscope enabling endoscopic observation inside a subject under radiation.

### SUMMARY OF THE INVENTION

A temperature measurement system according an aspect of the present invention includes an observation optical system, a branching unit configured to branch light from the observation optical system into first light including visible light from a subject and second light for temperature measurement, an image pickup device configured to receive the first light from the branching unit to pick up an image of the subject, a spectroscope configured to receive the second light from the branching unit and measure a plurality of spectral radiances of the subject, and a processor configured to calculate the temperature of the subject based on an output signal including the plurality of spectral radiances of the spectroscope.

An endoscope of an aspect of the present invention includes an observation optical system, a branching unit configured to branch light from the observation optical system into first light including visible light from a subject and second light for temperature measurement, and an image pickup device configured to receive the first light from the branching unit and pick up an image of the subject.

### BRIEF DESCRIPTION OF THE DRAWINGS

FIG. 1 is a configuration diagram showing a configuration of an endoscope system according to an embodiment of the present invention;

FIG. 2 is a diagram showing a configuration of a relay optical system according to the embodiment of the present invention;

FIG. 3 is a configuration diagram showing a configuration of an endoscope system according to a first modification of the present invention;

FIG. 4 is a cross-sectional view of an extensible insertion portion according to a second modification of the present invention;

FIG. 5 is a cross-sectional view of an endoscope having an insertion portion that is capable of changing an observation direction to an oblique front side according to a third modification of the present invention;

FIG. 6 is a configuration diagram showing a configuration of an endoscope system according to a fourth modification of the present invention;

FIG. 7 is a perspective view of one split optical fiber bundle according to a fourth modification of the present invention when an optical fiber bundle is split into two optical fiber bundles in an axial direction of the insertion portion;

FIG. 8 is a diagram showing a method of providing two pipe sleeves together at a distal end portion and a proximal end portion of the optical fiber bundle by combining the two split bundles according to the fourth modification of the present invention;

FIG. 9 is a partial perspective view of an optical fiber bundle having four grooves in the axial direction of the insertion portion on an outer peripheral portion of the optical fiber bundle according to the fourth modification of the present invention;

FIG. 10 is a diagram showing a configuration example of an endoscope system having a lighting unit according to the fourth modification of the present invention;

FIG. 11 is a perspective view of an optical fiber bundle in which the proximal end portion is branched into two parts according to the fourth modification of the present invention;

FIG. 12 is a configuration diagram showing a configuration of an endoscope system according to a fifth modification of the present invention;

FIG. 13 is a diagram showing a configuration of a switching device according to the fifth modification of the present invention;

FIG. 14 is a configuration diagram of an endoscope system according to a sixth modification of the present invention;

FIG. 15 is a configuration diagram of a controller according to the sixth modification of the present invention;

FIG. 16 is a perspective view showing a drive mechanism of a distal end portion of a probe according to the sixth modification of the present invention;

FIG. 17 is a diagram showing a configuration of an endoscope system having a detachable lighting unit according to a seventh modification of the present invention;

FIG. 18 is a configuration diagram of a temperature measurement system according to an eighth modification of the present invention;

FIG. 19 is a configuration diagram of an endoscope system according to a ninth modification of the present invention; and

FIG. 20 is a configuration diagram of an endoscope system according to a tenth modification of the present invention.

### DETAILED DESCRIPTION OF THE PREFERRED EMBODIMENT(S)

An embodiment according to the present invention will be described hereinafter with reference to the drawings.

### (Configuration)

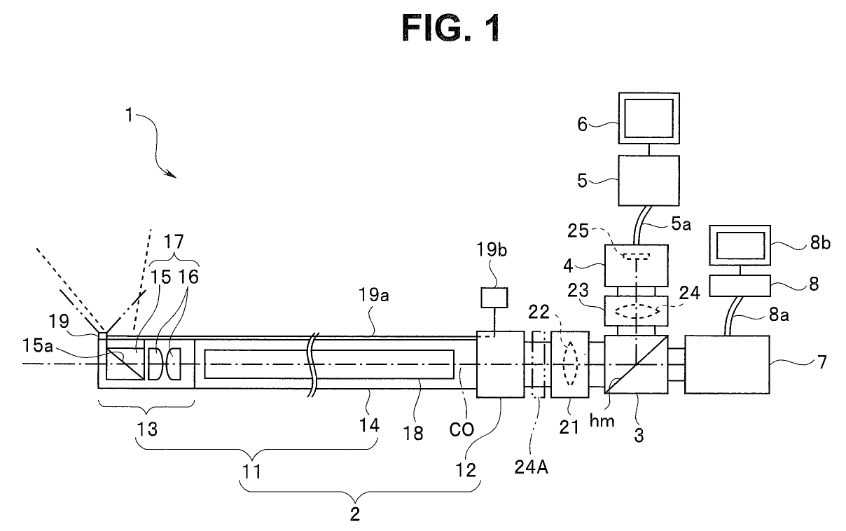

FIG. 1 is a configuration diagram showing a configuration of an endoscope system according to an embodiment.

An endoscope system 1 is a temperature measurement system including an endoscope 2, a beam splitter 3, an image pickup unit 4, an image processing unit 5, a monitor 6, a spectroscope 7, and a personal computer (hereinafter abbreviated as PC) 8.

The endoscope 2 is a rigid endoscope, and includes an elongated insertion portion 11 and an eyepiece unit 12. The eyepiece unit 12 is connected to the proximal end portion of the insertion portion 11. The endoscope 2 is, for example, a borescope, and is an observation device configured to observe the inside of a subject by inserting the insertion portion 11 from an opening portion provided in the subject.

The insertion portion 11 has a distal end portion 13 provided at the distal end of the insertion portion 11 and a relay lens portion 14 provided on the proximal end side of the distal end portion 13.

At the distal end portion 13, a prism 15 and plural lenses 16 are provided inside a distal end rigid member (not shown). The prism 15 and the plural lenses 16 configure an objective optical system 17. The prism 15 has a reflecting surface 15a, and deflects light from the subject to the plural lenses 16. The reflecting surface 15a of the prism 15 is inclined by only a predetermined angle with respect to a center axis CO of the insertion portion 11. The reflecting surface 15a deflects light from an oblique front side of the distal end portion 13 so that the light propagates in the optical axis direction of the plural lenses 16. The predetermined angle is set to, for example, an angle between 60 degrees and 70 degrees. Therefore, here, the endoscope 2 is a perspective-view type endoscope.

A relay optical system 18 is incorporated in the insertion portion 11. The objective optical system 17 and the relay optical system 18 configure an observation optical system.

A light emitting element 19 is arranged as a lighting unit in the distal end portion 13. The light emitting element 19 is connected to a power supply 19b via a signal line 19a. As indicated by the two-dotted chain line in FIG. 1, the light emitting element 19 emits illumination light to the object.

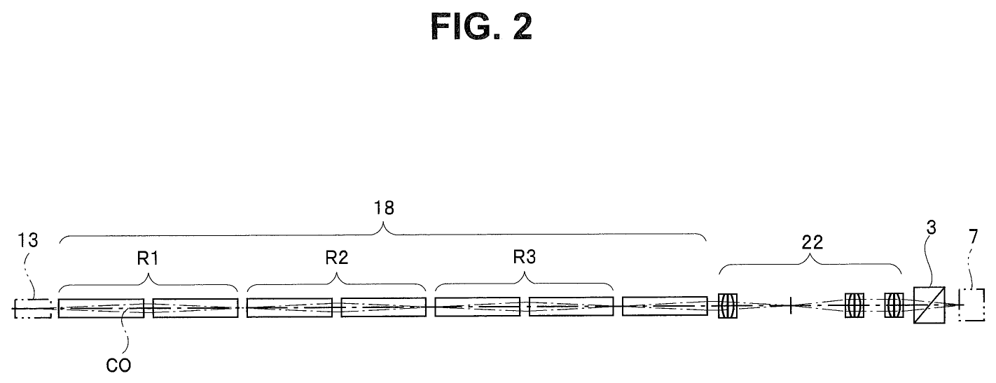

FIG. 2 is a diagram showing a configuration of the relay optical system 18. The relay optical system 18 includes plural lenses, and in this case, the relay optical system 18 is an optical system of three-time relay, and includes a first relay portion R1, a second relay portion R2, and a third relay portion R3.

The insertion portion 11 has a tubular member formed of metal, for example, stainless steel, and the prism 15, the plural lenses 16 and the relay optical system 18 are arranged in the tubular member. In order to secure the heat resistance of the endoscope 2, each optical member configuring the observation optical system is held in the tubular member by a spring member or the like.

In order to enable the endoscope 2 of the endoscope system 1 to be used under a radiation environment or a high temperature environment, quartz glass is used for each optical member configuring the observation optical system. The quartz glass has radiation resistance and has heat resistance up to 1600° C.

The refractive index of the quartz glass is equal to 1.46 and the critical angle of the quartz glass is equal to about 42 degrees. Therefore, it is possible to secure the total reflection of the reflecting surface 15a of the prism 15 by using a perspective-view type endoscope configured to observe in an obliquely forward direction rather than an endoscope configured to observe in a side viewing direction orthogonal to the center axis CO (that is, the optical axis of the relay lens portion) of the insertion portion 11.

Returning to FIG. 1, the eyepiece unit 12 and the beam splitter 3 are connected to each other via an adapter 21. An optical system 22 for color correction is provided in the adapter 21. The optical system 22 for color correction is formed of multi-component glass.

The optical system 22 for color correction serves as a chromatic aberration correction unit configured to correct chromatic aberration and spherical aberration occurring in the objective optical system and the relay optical system which are formed of quartz glass. In this case, the optical system 22 for color correction is a correction optical system of 1.5-time relay as shown in FIG. 2.

In other words, the chromatic aberration correction unit configured to correct chromatic aberration occurring in the observation optical system is arranged between the observation optical system and the beam splitter 3.

Note that when the relay optical system 18 is an optical system of one-time relay, a correction optical system of 0.5-time relay is used as the optical system 22 for color correction.

The beam splitter 3 includes a half mirror hm. The half mirror hm transmits a part of light from the eyepiece unit 12 and reflects the rest of the light. The light reflected by the half mirror hm is emitted to the image pickup unit 4.

The beam splitter 3 and the image pickup unit 4 are connected to each other via an adapter 23. An optical system 24 for focus adjustment is provided in the adapter 23. The optical system 24 for focus adjustment includes a lens configured to adjust the focal position to the image pickup unit 4.

A user can perform focus adjustment on an object image from the observation optical system by turning an adjustment ring (not shown).

Note that the driving of the lens of the optical system for focus adjustment may be performed by electrical driving.

As indicated by a two-dotted chain line in FIG. 1, an adapter 24A may be arranged between the eyepiece unit 12 and the adapter 21, and the optical system 24 for focus adjustment may be provided in the adapter 24A.

The image pickup unit 4 includes an image pickup device 25. The image pickup unit 4 is connected to the image processing unit 5 via a cable 5a.

The image processing unit 5 drives the image pickup device 25 and receives an image pickup signal from the image pickup device 25. The image processing unit 5 performs image processing on the received image pickup signal to generate an endoscopic image. The image processing unit 5 outputs the generated image signal of the endoscopic image to the monitor 6. As a result, an endoscopic image of the inside of the subject obtained by the endoscope 2 is displayed on the monitor 6.

Light is passed through the half mirror hm and supplied from the beam splitter 3 to the spectroscope 7. In other words, the beam splitter 3 receives light from the observation optical system and splits the received light into first light and second light, the image pickup device 25 receives the first light from the beam splitter 3, and the spectroscope 7 receives the second light from the beam splitter 3.

The spectroscope 7 is a device configured to measure plural spectral radiances of light transmitted through the half mirror hm. As described later, signals of the plural spectral radiance values measured by the spectroscope 7 are supplied to the PC 8 as output signals. The PC 8 is a processor configured to calculate the temperature of the subject from the plural spectral radiance values. Here, the spectroscope 7 outputs two spectral radiance values, and the PC 8 which is a processor having hardware configures a temperature calculating section configured to calculate the temperature of the subject from the two spectral radiance values.

The PC 8 includes a central processing unit (CPU), ROM, RAM, and the like, and a software program for calculating the temperature of the subject is stored in ROM or the like. The CPU reads out and executes the software program stored in the ROM, whereby the PC 8 can realize various functions such as temperature calculation.

Note that the processor may be an electronic circuit configured to perform various operations such as temperature calculation of the subject, or a circuit such as an FPGA (field programmable gate array).

The light transmitted through the half mirror hm is light in the whole or partial region of the endoscopic image displayed on the monitor 6. The light transmitted through the half mirror hm is incident on the spectroscope 7. The spectroscope 7 measures two spectral radiances of the region corresponding to the endoscopic image displayed on the monitor 6.

The spectroscope 7 and the PC 8 are connected to each other by a cable 8a. A monitor 8b is connected to the PC 8.

In the PC 8, a predetermined software program is executed to calculate the temperature of the subject from the output signal from the spectroscope 7. The software program configures a temperature calculating section. The PC 8 displays information on the calculated temperature value on the monitor 8b.

Therefore, the observation optical system, the spectroscope 7 and the PC 8 configure a radiation thermometer, in this case, a two-color thermometer.

As described above, the endoscope system 1 is a temperature measurement system. The beam splitter 3 configures a branching unit configured to branch light from the observation optical system into first light including visible light from the subject and second light for temperature measurement. The image pickup device 25 receives the first light from the beam splitter 3 to pick up an image of the subject. The spectroscope 7 receives the second light from the beam splitter 3 to measure plural spectral radiances of the subject. The PC 8 configures a temperature calculating section configured to calculate the temperature of the subject based on an output signal including the plural spectral radiance values of the spectroscope 7.

### (Operation)

Next, the operation of the above-described endoscope system 1 will be described.

A user who is an examiner inserts the insertion portion 11 into the subject and approaches the distal end portion 13 to a site to be examined in the subject, and when the distance from the distal end portion 13 to the surface of the subject reaches a predetermined distance, an endoscopic image of the site is displayed on the monitor 6. Therefore, by approaching the distal end portion 13 to a desired site, the user can watch the endoscopic image displayed on the monitor 6 and perform an endoscopic examination of the subject.

At the same time, the temperature of a site or region of the subject included in the endoscopic image is displayed on the monitor 8b, and the user can check the temperature of the site or region of the subject included in the endoscopic image. When the user changes the position of the distal end portion 13, the temperature of a site or region of the subject included in the changed endoscopic image is displayed on the monitor 8b.

In the endoscope system 1, an endoscopic image of the subject is generated from an object image obtained by the observation optical system and displayed on the monitor 6, a part of light from the observation optical system is branched, plural spectral radiances are measured by the spectroscope 7, and the temperature of the subject calculated from the measured plural spectral radiances is displayed on the monitor 8b.

Therefore, the user can also know the temperature of a site of the subject being observed by using the endoscope 2 while watching the image of the site of the subject.

As described above, according to the above-described embodiment, it is possible to provide a temperature measurement system and an endoscope that can acquire an image of a subject and can measure the temperature of a desired site of the subject in a noncontact manner.

Particularly, according to the above-described embodiment, it is possible within a subject under a high-temperature environment such as an industrial furnace or an engine to perform endoscopic observation of a desired site in the subject and measure the temperature of the site in a noncontact manner.

Furthermore, since quartz glass is used for the observation optical system, it is possible to perform endoscopic observation and temperature measurement on a desired site in the subject even under a radiation environment.

Note that in the above-described endoscope system 1, the endoscopic image is generated in the image processing unit 5, but the output of the image pickup unit 4 may be supplied to the PC 8 to perform temperature calculation processing and image generation processing in the PC 8. In this case, calculated temperature information and generated endoscopic image are displayed on the monitor 8b.

The above-described endoscope 2 is a perspective-view type endoscope suitable for examination of a pipe, etc., but may be a front-view type endoscope configured to observe the insertion direction of the insertion portion 11. In that case, the prism 15 is not provided to the distal end portion 13.

In the above-described embodiment, the relay optical system 18 arranged in the tubular member of metal such as stainless steel is used, but light from the object may be transmitted to the beam splitter 3 with an optical fiber bundle obtained by bundling plural thin optical fibers. In that case, since the thin optical fiber bundle has flexibility, the endoscope 2 becomes a flexible endoscope. The optical fiber is a quartz glass fiber when radiation resistance and heat resistance are considered, but the optical fiber is a glass fiber which is not quartz glass when neither radiation resistance nor heat resistance is considered.

Next, modifications of the above-described endoscope system will be described.

Note that in each of the modifications described below, the same reference signs are given to the same components as the respective components of the endoscope system 1 according to the above-described embodiment, and description on the components will be omitted. In each modification, the same reference signs are given to the same components as the components of the other modifications, and description on the components will be omitted.

### (First Modification)

The above-described embodiment is a temperature measurement system using an endoscope having a perspective-view type endoscope (or a front-view type endoscope), but the present first modification is a temperature measurement system having a stereoscopic endoscope.

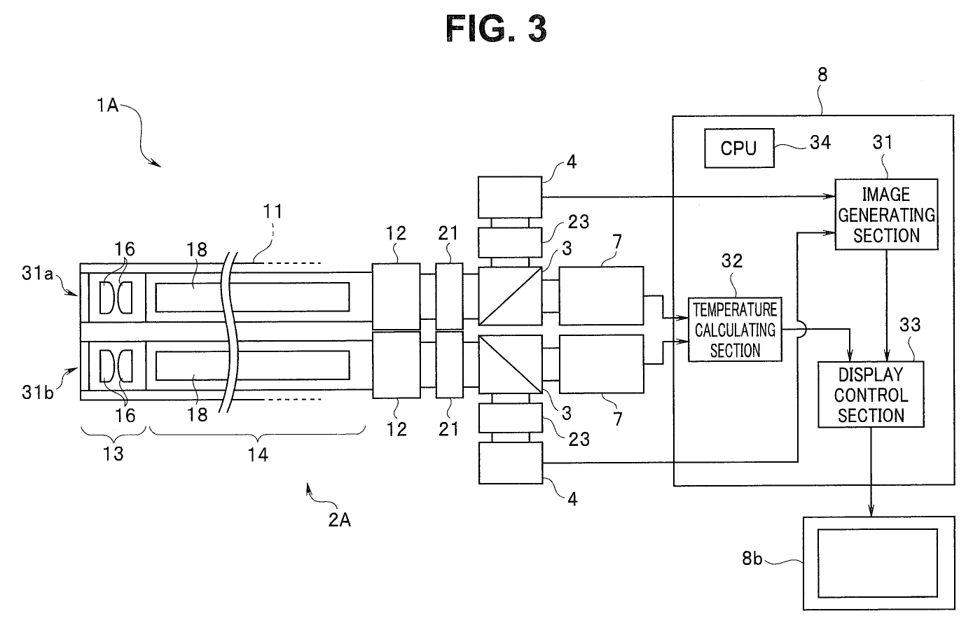

FIG. 3 is a configuration diagram showing a configuration of an endoscope system according to the first modification.

The endoscope system 1A includes an endoscope 2A, and the endoscope 2A is a temperature measurement system having two observation windows 31a and 31b at the distal end portion 13. An observation optical system including plural lenses 16 and a relay optical system 18 is arranged in the insertion portion 11 so as to correspond to each of the observation windows 31a and 31b. Therefore, the two observation optical systems are arranged with a predetermined parallax.

An eyepiece unit 12 is provided on a proximal end side of each relay optical system 18. A beam splitter 3 is connected to each eyepiece unit 12 via an adapter 21.

Each beam splitter 3 includes a half mirror hm. Each half mirror hm transmits a part of light from the eyepiece unit 12 and reflects the rest of the light. The light reflected by each half mirror hm is emitted to an image pickup unit 4.

Light passing through each half mirror hm is supplied from the beam splitter 3 to a spectroscope 7.

Output signals of the two image pickup units 4 and output signals of the two spectroscopes 7 are supplied to PC 8.

As described above, the endoscope system 1A includes two observation optical systems, two beam splitters 3, two image pickup devices 25 and two spectroscopes 7.

The PC 8 includes an image generating section 31, a temperature calculating section 32, and a display control section 33. The image generating section 31, the temperature calculating section 32 and the display control section 33 are software programs, and stored in a memory (not shown). The PC 8 has a central processing unit (CPU) 34, and reads out and executes the respective programs of the image generating section 31, the temperature calculating section 32, and the display control section 33 from the memory.

The image generating section 31 generates a 3D image from images obtained by the two image pickup devices 25. The temperature calculating section 32 calculates two temperatures from the two output signals from the two spectroscopes 7, and calculates an average value of the two temperatures obtained by the calculation.

The display control section 33 generates a display image for displaying the 3D image and the temperature information on the monitor 8b, and outputs an image signal of the display image to the monitor 8b.

Therefore, according to the first modification, it is possible to provide an endoscope system configured to measure the temperature of a site of a subject being observed by the endoscope 2 in a noncontact manner and report the temperature to the user even in the case of the stereoscopic endoscope.

### (Second Modification)

In the endoscope of the above-described embodiment, the length of the insertion portion is constant. However, in the present second modification, the length of the insertion portion is finely adjustable.

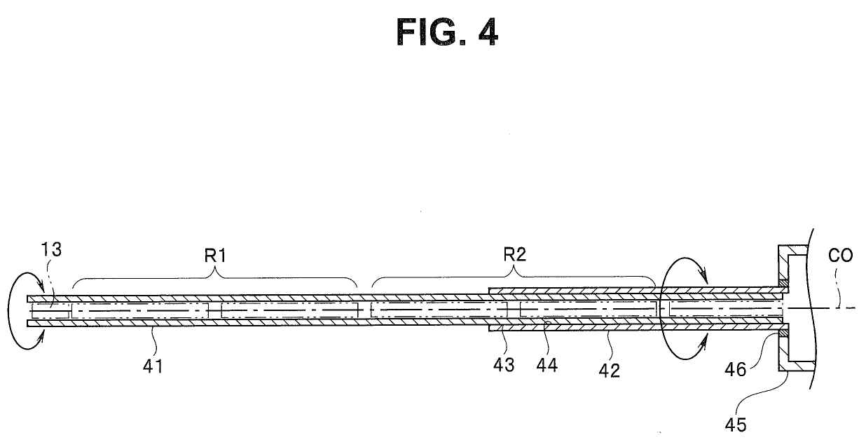

FIG. 4 is a cross-sectional view of the extensible insertion portion according to the second modification. The insertion portion 11 includes plural relay portions, and in this case, a relay optical system including first and second relay portions R1 and R2 is provided in a first tubular member 41. Light emitted from the relay optical system has been collimated.

An outer peripheral portion on the proximal end side of the first tubular member 41 is screwed with an inner peripheral surface of the second tubular member 42. A male thread portion 43 is formed on an outer peripheral surface on the proximal end side of the first tubular member 41, and a female thread portion 44 is formed on an inner peripheral surface on the distal end side of the second tubular member 42. The user can turn the first tubular member 41 around the center axis of the insertion portion 11 with respect to the second tubular member 42. Therefore, by turning the first tubular member 41, the user can advance and retract the first tubular member 41 in the direction of the center axis CO with respect to the second tubular member 42. In other words, the male thread portion 43 and the female thread portion 44 configure an advancing/retracting mechanism of the first tubular member 41.

The proximal end portion of the second tubular member 42 is connected to a case 45 of the eyepiece unit 12. The proximal end portion of the second tubular member 42 is connected to the case 45 via a bearing 46 so as to be rotatable around the center axis CO. The center axis of the tubular member 41 and the center axis of the second tubular member 42 coincide with the center axis CO of the insertion portion 11. Therefore, the user can turn the second tubular member 42 around the center axis CO by turning the second tubular member 42. The tubular member 41 is turned together with the second tubular member 42 around the center axis CO. In other words, the bearing 46 configures a turning mechanism of the second tubular member 42.

As described above, since the endoscope 2 has a mechanism capable of advancing and retracting an emission position of collimated light from the relay optical system in the direction of the center axis CO, the user can finely adjust the position of the distal end portion 13 of the endoscope 2 with respect to the subject.

Since the endoscope 2 has a mechanism capable of turning the insertion portion 11 around the center axis CO, the user can also finely adjust the angle of the distal end portion 13 of the endoscope 2 around the center axis CO.

Therefore, according to the second modification, in addition to the effect of the above-described embodiment, the user can finely adjust the distance to an observation site and the angle of the observation site around the observation optical system after positioning the endoscope 2 in the subject, so that an effect of making the position adjustment of the distal end portion 13 easy can be achieved.

### (Third Modification)

The endoscope of the above-described embodiment is a perspective-view type endoscope, and the observation direction to the oblique front side is a certain direction from a predetermined position on the center axis CO of the insertion portion 11. The perspective-view type endoscope of the present third modification is capable of changing the observation direction to the oblique front side.

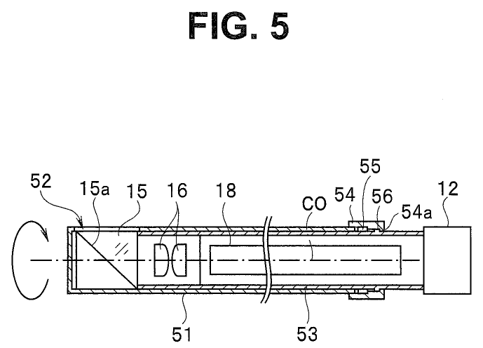

FIG. 5 is a cross-sectional view of an endoscope having an insertion portion capable of changing the observation direction to the oblique front side according to the third modification.

A prism 15 of the distal end portion 13 is arranged in a tubular member 51 the distal end of which is closed. The tubular member 51 has an opening portion 52 through which light is incident on the prism 15.

The plural lenses 16 and the relay optical system 18 are arranged in the tubular member 53, and the proximal end portion of the tubular member 53 is fixed to the eyepiece unit 12.

The distal end portion of the tubular member 53 is inserted from the proximal end portion of the tubular member 51 into the tubular member 51. The tubular member 53 is inserted into the tubular member 51 so as to be turnable around the center axis CO in the tubular member 51. A retaining ring 54 is fixed to the proximal end portion of the tubular member 51. A bearing 55 is fixed to the inner peripheral surface of the retaining ring 54.

An inwardly facing flange portion 54a is formed on a proximal end side of the retaining ring 54. The inwardly facing flange portion 54a abuts against a peripheral protrusion portion 56 formed on the outer peripheral portion of the tubular member 53.

The tubular member 51 is turnable around the center axis CO with respect to the tubular member 53 by the bearing 55.

Therefore, according to the third modification, in addition to the effect of the above-described embodiment, the user can change the observation direction around the center axis CO of the endoscope 2 by turning the tubular member 51 around the center axis CO.

### (Fourth Modification)

In the above-described embodiment, a part of light passing through the relay optical system is branched in the beam splitter and input to the spectroscope. In the present modification, light from the subject is led to the image pickup unit by using an optical fiber bundle, and a part of the light from the subject is led to the spectroscope by using a part of the optical fiber bundle.

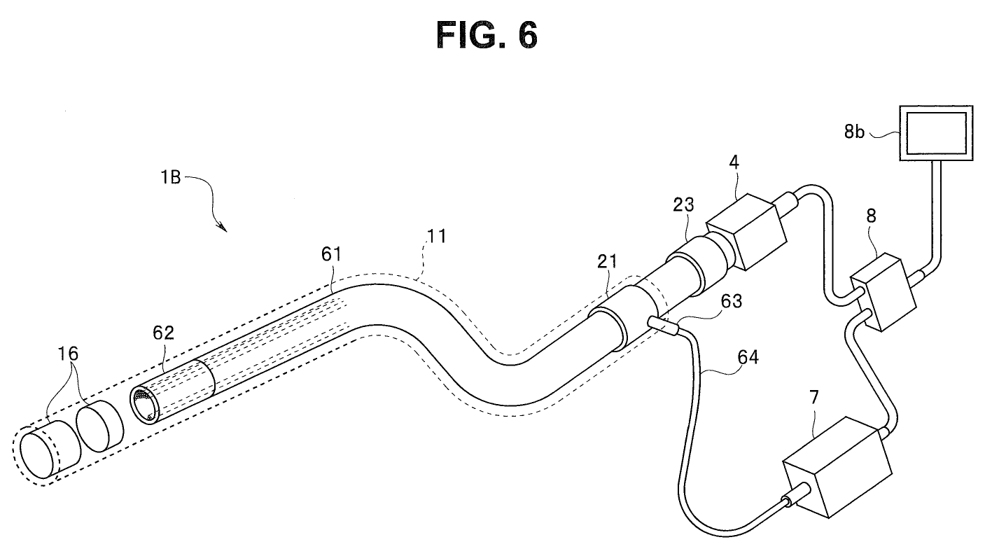

FIG. 6 is a configuration diagram showing a configuration of an endoscope system according to a fourth modification. An endoscope 2 used in the endoscope system 1B of the present modification is a front-view type endoscope.

Plural lenses 16 are arranged at the distal end portion 13 of the insertion portion 11 of the endoscope 2. A distal end portion of an optical fiber bundle 61 including plural optical fibers is arranged at the proximal end portion of the plural lenses 16, and the optical fiber bundle 61 is inserted through an inside of an envelope (not shown) of the insertion portion 11.

The plural lenses 16 may be provided in an optical adapter to be attached to the distal end of the insertion portion 11.

The distal end portion of the optical fiber bundle 61 is provided with a cylindrical pipe sleeve 62.

The proximal end portion of the optical fiber bundle 61 is fixed to an adapter 21. Some optical fibers of the optical fiber bundle 61 are inserted from the branching unit 63 of the adapter 21 through an inside of a cable 64. The proximal end portions of the plural optical fibers in the cable 64 are connected to the spectroscope 7.

Plural optical fibers at predetermined positions on the distal end surface of the optical fiber bundle 61 are selected as the plural optical fibers branched into the cable 64. In this case, as described later, the predetermined positions are two places in the vicinity of a center portion of the distal end surface of the optical fiber bundle 61.

Note that the predetermined positions may be, for example, plural positions evenly distributed on the distal end surface of the optical fiber bundle 61, or may be a circular center position on the distal end surface of the optical fiber bundle 61.

The remaining plural optical fibers excluding the plural optical fibers branched into the cable 64 are connected to the image pickup unit 4 via the adapter 23. The image pickup unit 4 is connected to the insertion portion 11 so that light emitted from the proximal end portions of the plural optical fibers hits the image pickup surface of the image pickup device 25 of the image pickup unit 4.

As described above, light from the object is transmitted to the image pickup unit 4 by the optical fiber bundle 61, and a part of the optical fiber bundle 61 is branched from the branching unit 63 and transmitted to the spectroscope 7. Therefore, the plural optical fibers branched into the cable 64 are some optical fibers selected from the optical fiber bundle 61, and light for temperature measurement of the subject is branched by the branched plural optical fibers.

The spectroscope 7 measures two spectral radiance values based on light from a part of the optical fiber bundle 61.

The image pickup unit 4 and the spectroscope 7 are connected to the PC 8. The PC 8 generates an endoscopic image from an image obtained by the image pickup unit 4, and calculates the temperature of the subject based on an output signal from the spectroscope 7. The PC 8 generates a display image, and outputs the display image to the monitor 8b. The display image is an image obtained by combining the endoscopic image and temperature information calculated based on the output signal from the spectroscope 7.

In other words, the optical fiber bundle 61 has a branching unit configured to branch light passing through a part of the optical fiber bundle 61 as light for image generation to the image pickup device 25, and branch light passing through another part of the optical fiber bundle 61 as light for temperature measurement to the spectroscope 7.

Therefore, an effect similar to the effect of the above-described embodiment can also be obtained by the endoscope system according to the fourth modification.

A method of branching, for example, optical fibers at two places on the distal end surface of the optical fiber bundle 61 in the above-described example of FIG. 6 will be described.

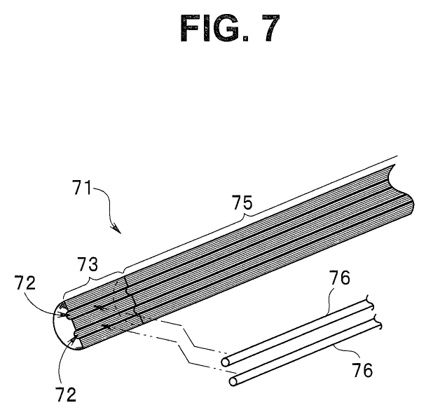

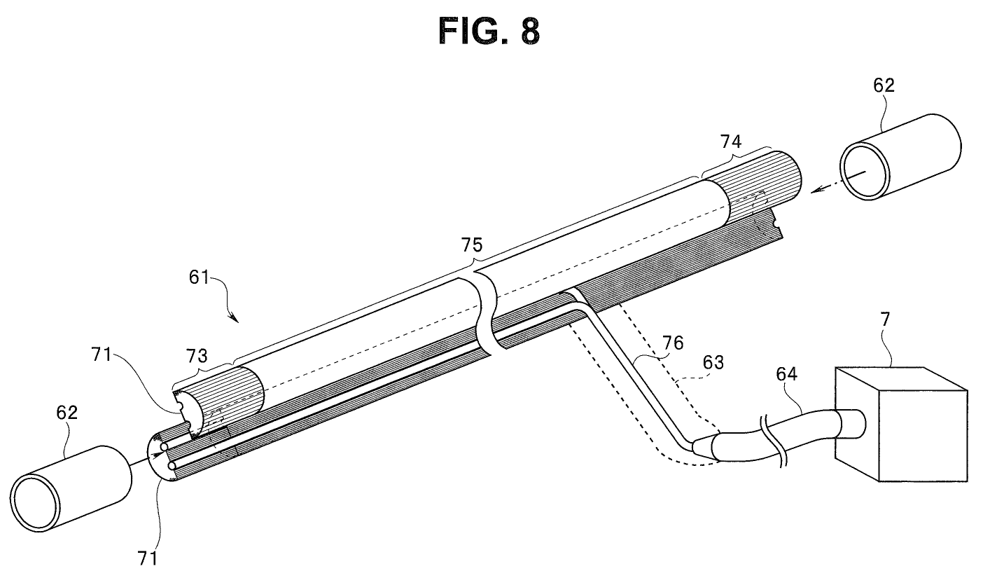

FIG. 7 is a perspective view of one split bundle when the optical fiber bundle 61 is split into two bundles in the axial direction of the insertion portion 11. FIG. 8 is a diagram showing a method of providing two pipe sleeves at the distal end portion and the proximal end portion of the optical fiber bundle 61 by combining the two split bundles.

As shown in FIG. 7, a semi-cylindrical optical fiber split bundle 71 is formed by using a predetermined jig (not shown) for plural optical fibers. A distal end portion of the split bundle 71 is welded with a resin. At this time, the distal end portion of the split bundle 71 is formed so that two groove portions 72 are formed at predetermined positions in the axial direction of the split bundle 71 on a semi-cylindrical flat portion.

Each groove portion 72 is arranged so that one optical fiber 76 or half of two or more optical fibers 76 to be branched to the spectroscope 7 enters the groove portion 72.

Therefore, the distal end portion 73 of the split bundle 71 is an optical fiber fixing portion at which plural optical fibers are fixed, and also the proximal end portion 74 of the split bundle 71 is an optical fiber fixing portion at which the plural optical fibers are fixed as shown in FIG. 8. An intermediate portion 75 between the distal end portion 73 and the proximal end portion 74 is an optical fiber non-fixing portion at which the plural optical fibers are not fixed.

After bringing the flat portions of the two split bundles 71 into close contact with each other, the distal end portions 73 and the proximal end portions 74 are covered with the pipe sleeves 62, respectively.

As shown in FIGS. 7 and 8, it is possible to specify and branch the optical fibers at the two places on the distal end surface of the optical fiber bundle 61.

In FIGS. 7 and 8, the two grooves are provided to each split bundle 71, and light in the vicinity of the center portion of the distal end surface of the optical fiber bundle 61 is led to the spectroscope 7. However, plural grooves may be provided on the outer peripheral surface of the optical fiber bundle 61 so that light at the outer peripheral portion of the distal end surface of the optical fiber bundle 61 is led to the spectroscope 7.

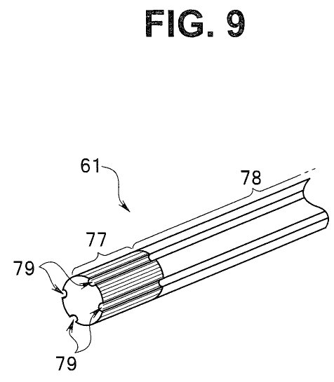

FIG. 9 is a partial perspective view of the optical fiber bundle 61 having four grooves in the axial direction of the insertion portion 11 on the outer peripheral portion of the optical fiber bundle 61. As shown in FIG. 9, a distal end portion 77 of the optical fiber bundle 61 is a fixing portion, and a proximal end portion 78 of the distal end portion 77 is a non-fixing portion. Although not shown, the proximal end portion of the optical fiber bundle 61 is also a fixing portion. Four grooves 79 are provided equally in the peripheral direction in the axial direction of the insertion portion 11 on the outer peripheral surface of the distal end portion 77.

Therefore, even the configuration shown in FIG. 9 makes it possible to specify and branch optical fibers at plural positions on the distal end surface of the optical fiber bundle 61.

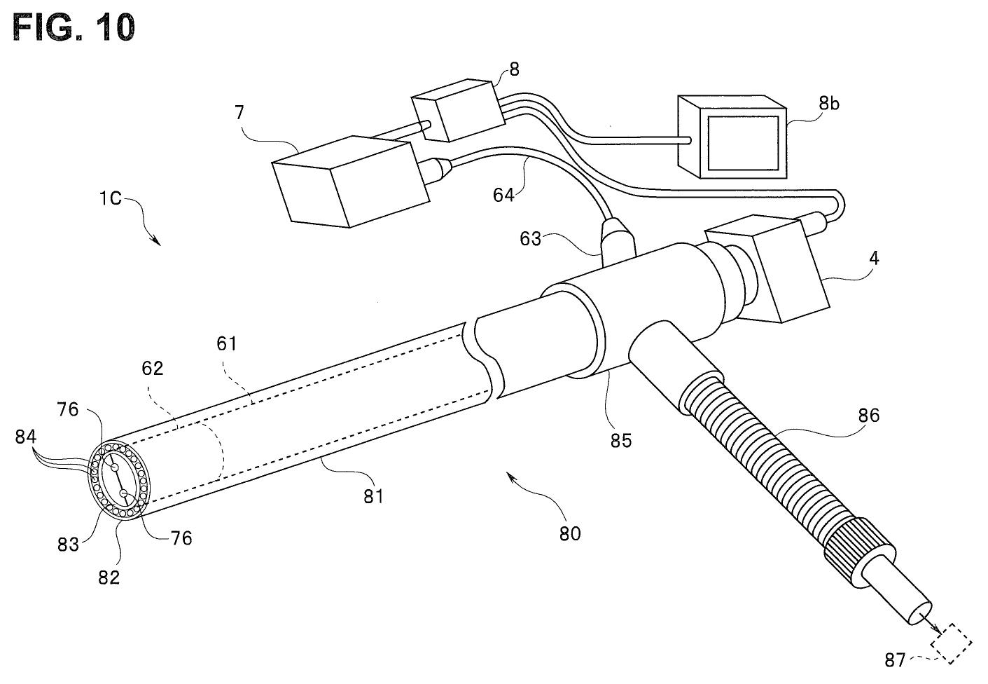

Note that when the endoscope system has a lighting unit, the endoscope system may have a configuration as shown in FIG. 10.

FIG. 10 is a diagram showing a configuration example of an endoscope system having a lighting unit.

The endoscope system 1C shown in FIG. 10 has a lighting unit 80. The lighting unit 80 has a double pipe 81. The double pipe 81 has an outer pipe 82 and an inner pipe 83. The outer diameter of the inner pipe 83 is smaller than the inner diameter of the outer pipe 82. The inner pipe 83 is inserted in the outer pipe 82. The optical fiber bundle 61 is inserted in the inner pipe 83.

A gap is formed between the outer peripheral surface of the outer pipe 82 and the inner peripheral surface of the inner pipe 83, and a light guide including plural optical fibers 84 is inserted in the axial direction of the double pipe 81 through the gap.

The proximal end portion of the double pipe 81 is provided with a light guide branching unit 85. The light guide including the plural optical fibers 84 branches off from the light guide branching unit 85 and is inserted through an inside of a light guide cable 86. As shown in FIG. 10, the distal end portions of the plural optical fibers 84 are arranged annularly along the outer peripheral surface of the optical fiber bundle 61, and the proximal end portions of the plural optical fibers 84 are connected to a light source device 87.

Light from the light source device 87 passes through the plural optical fibers 84 and exits from the distal end surface of the double pipe 81.

Therefore, the user can perform endoscopic examination and temperature measurement even at a dark place.

Next, a configuration of the lighting unit in which the plural optical fibers are uniformly dispersed on the distal end surface of the optical fiber bundle 61, for example, in the above-described example of FIG. 6 will be described.

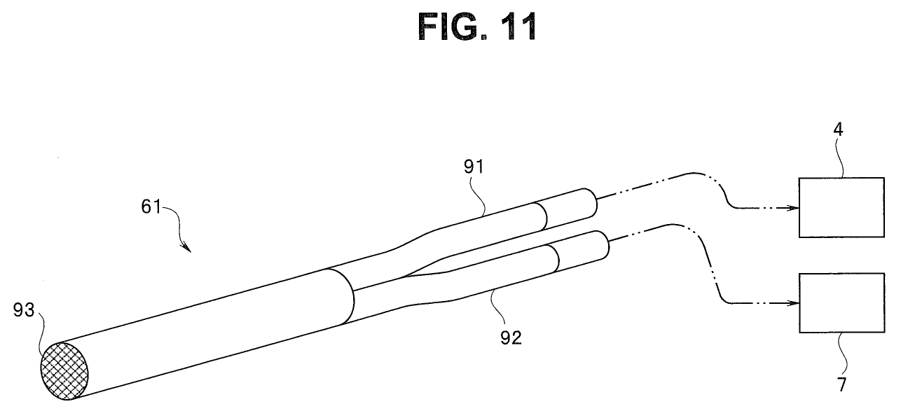

FIG. 11 is a perspective view of an optical fiber bundle in which a portion on the proximal end side is branched into two bundles.

A half optical fiber group 91 included in the optical fiber bundle 61 is connected to the image pickup unit 4, and a remaining half optical fiber group 92 is connected to the spectroscope 7. The optical fiber group 92 configures the branching unit.

The plural optical fibers of the optical fiber group 91 and the plural optical fibers of the optical fiber group 92 are arranged to be equally dispersed on the distal end surface 93 of the optical fiber bundle 61. In other words, the plural optical fibers of the optical fiber group 91 receive light incident on the entire surface of the distal end surface 93 of the optical fiber bundle 61, and transmit the light to the image pickup unit 4. Similarly, the plural optical fibers of the optical fiber group 92 receive light incident on the entire surface of the distal end surface 93 of the optical fiber bundle 61 and transmit the light to the spectroscope 7.

Therefore, the image pickup unit 4 can receive light from the objective optical system 17 (not shown), and an endoscopic image of the subject is generated in the PC 8 or the like. The spectroscope 7 can receive light from the objective optical system 17, and the temperature in the same region as the endoscopic image of the subject is calculated in the PC 8.

Therefore, even the configuration shown in FIG. 11 makes it possible to specify and branch plural optical fibers corresponding to the entire surface of the distal end surface of the optical fiber bundle 61.

### (Fifth Modification)

The endoscope system shown in FIG. 10 according to the fourth modification has a light guide dedicated to illumination. However, in an endoscope system according to the fifth modification, the optical fiber for temperature measurement is also used as a light guide for illumination light.

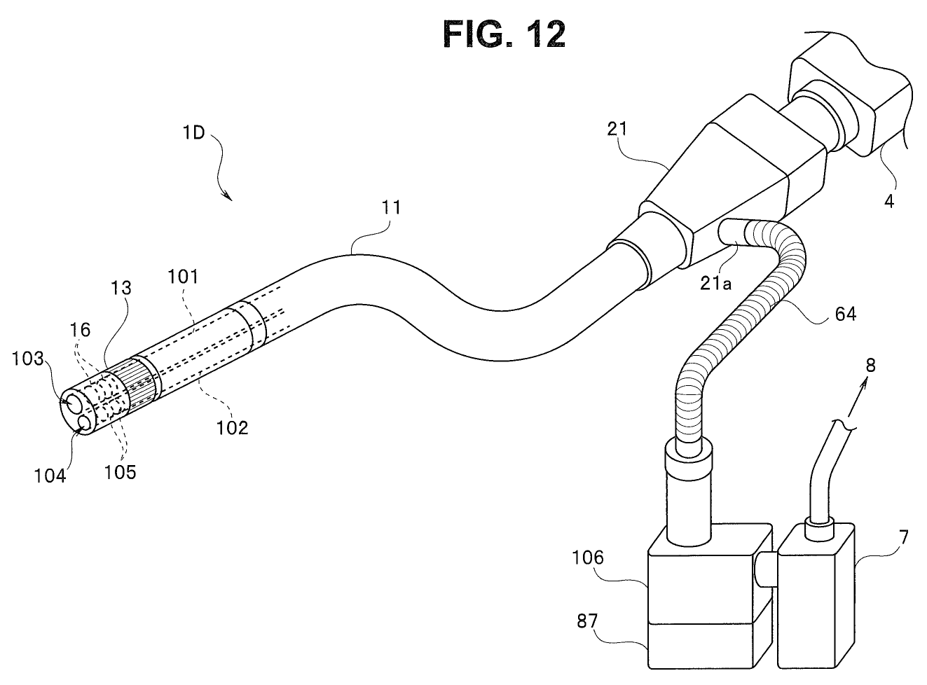

FIG. 12 is a configuration diagram showing a configuration of an endoscope system according to a fifth modification. An endoscope 2 of the present modification is a front-view type endoscope.

In the endoscope system 1D, two optical fiber bundles are inserted in an insertion portion 11 of the endoscope. A first optical fiber bundle 101 and a second optical fiber bundle 102 are inserted in the insertion portion 11. In other words, an observation optical system includes the first optical fiber bundle 101 having plural optical fibers and the second optical fiber bundle 102 having plural optical fibers. The first optical fiber bundle 101 irradiates the image pickup device 25 with light for image generation.

A first observation window 103 and a second observation window 104 are provided on the distal end surface of the distal end portion 13. Plural lenses 16 configuring the observation optical system are arranged behind the first observation window 103. The distal end surface of the first optical fiber bundle 101 is arranged behind the plural lenses 16.

Plural lenses 105 are arranged behind the second observation window 104. The distal end surface of the second optical fiber bundle 102 is arranged behind the plural lenses 105.

The proximal end portion of the first optical fiber bundle 101 is connected to the image pickup unit 4 via the adapter 21.

The second optical fiber bundle 102 is inserted in a cable 64 branched at the branching unit 21a of the adapter 21. The proximal end portion of the cable 64 is connected to a switching unit 106.

The switching unit 106 is connected to the light source device 87 and the spectroscope 7.

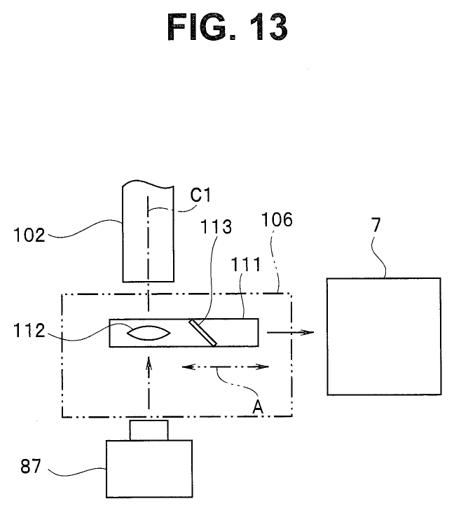

FIG. 13 is a diagram showing the configuration of the switching unit 106.

The switching unit 106 has a movable member 111. The movable member 111 is provided with a condensing lens 112 and a reflection mirror 113. The movable member 111 is movable in a direction indicated by an arrow A of a two-dotted chain line by a drive unit such as a motor (not shown) or by a user's manual operation.

The movable member 111 can take two positions. A first position is a position where the condensing lens 112 is placed on an optical axis C1 of the second optical fiber bundle 102. The second position is a position where the reflection mirror 113 is placed on the optical axis C1 of the second optical fiber bundle 102. FIG. 13 shows a case where the movable member 111 is located at the first position.

When the movable member 111 is located at the first position, light from the light source device 87 is focused on the proximal end surface of the second optical fiber bundle 102 by the condensing lens 112. Therefore, the light of the light source device 87 is emitted as illumination light from the observation window 104, and reflected light from the subject is incident on the observation window 103. Light from the subject which is incident on the observation window 103 passes through the first optical fiber bundle 101 and is supplied to the image pickup unit 4, whereby an endoscopic image can be generated in the PC 8.

When the movable member 111 is located at the second position, light from the proximal end surface of the second optical fiber bundle 102 is directed to the spectroscope 7 by the reflection mirror 113. Therefore, upon receiving the light from the subject, the spectroscope 7 can measure plural spectral radiances of the subject.

In other words, the switching unit 106 is switchable between a first state and a second state, and the switching unit 106 is provided on the proximal end side of the second optical fiber bundle 102 to emit the light for temperature measurement to the spectroscope 7 under the first state while emitting the illumination light from the light source device 87 to the proximal end portion of the second optical fiber bundler 102 under the second state.

According to the endoscope system 1D, when the subject is bright, the user can simultaneously perform the observation of the endoscopic image of the subject and the temperature measurement of the subject by placing the movable member 111 at the second position.

When the subject is dark, the user places the movable member 111 at the first position to irradiate the subject with the illumination light and observe the subject with the endoscopic image displayed on the monitor 8b. When it is desired to know the temperature of a subject appearing in an endoscopic image during endoscopic observation, the movable member 111 is placed at the second position, whereby the spectroscope 7 receives light from the subject and the PC 8 calculates the temperature of the subject. As a result, temperature information of the subject is displayed on the monitor 8b although the endoscopic image is not displayed on the monitor 8b in real time.

### (Sixth Modification)

In the fifth modification, the optical fiber bundles for the temperature measurement and the illumination light are provided in the insertion portion 11. However, in the present sixth modification, the optical fiber bundles for the temperature measurement and the illumination light are arranged in a probe which is insertable into a treatment instrument insertion channel formed in the insertion portion 11.

Furthermore, the operation of the movable member 111 is interlocked with the image display operation, whereby it is possible to perform temperature display while displaying an image when just previous illumination is turned on when illumination is turned off. This configuration makes it possible to display the temperature while displaying the image.

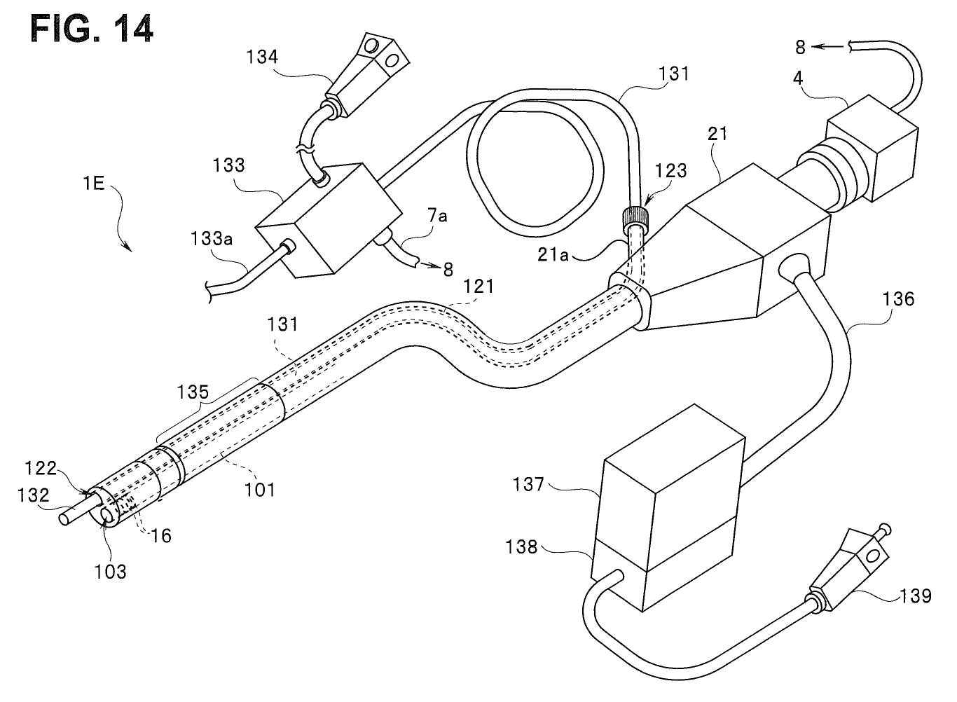

FIG. 14 is a configuration diagram of an endoscope system according to a sixth modification. An endoscope 2 of the present modification is a front-view type endoscope.

A treatment instrument insertion channel 121 is formed in parallel with the center axis CO of the insertion portion 11 in the insertion portion 11 of the endoscope of the endoscope system 1E. An opening portion 122 of the treatment instrument insertion channel 121 is formed on the distal end surface of the distal end portion 13. An opening portion 123 on the proximal end side of the treatment instrument insertion channel 121 is provided in the branching unit 21a of the adapter 21 connected to the proximal end portion of the insertion portion 11.

A probe 131 including plural optical fibers is allowed to be inserted into the opening portion 123. When the user pushes or pulls the probe 131 at the opening portion 123, the distal end portion 132 of the probe 131 can protrude from or retract into the opening portion 122.

The proximal end portion of the probe 131 is connected to a controller 133. A remote controller 134 is connected to the controller 133. The controller 133 is supplied with power from a power supply line 133a.

The optical fiber bundle 101 configures an observation optical system. The optical fiber bundle 101 irradiates the image pickup device 25 of the image pickup unit 4 with light from the subject. The observation optical system is arranged inside the insertion portion 11 of the endoscope 2, and the probe 131 is arranged inside a channel provided in the insertion portion 11.

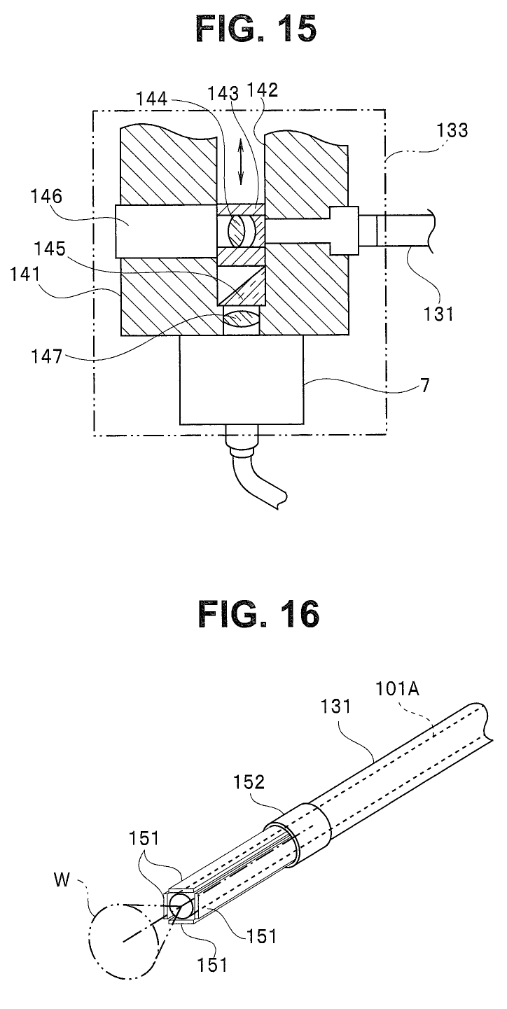

FIG. 15 is a configuration diagram of the controller 133.

The controller 133 has a support member 141 provided in a housing. The support member 141 is made of, for example, metal, and a hole 142 is formed in the support member 141.

A movable member 143 is arranged in the hole 142. The movable member 143 is movable in a predetermined direction indicated by an arrow in the hole 142 by a drive mechanism such as a motor (not shown).

The movable member 143 is provided with a condensing lens 144 including plural lenses and a prism 145. Therefore, the condensing lens 144 and the prism 145 move together in the hole 142 in connection with movement of the movable member 143.

The light source device 146 and the spectroscope 7 are fixed to the support member 141. The light source device 146 includes a light emitting element such as an LED (light emitting diode) and emits illumination light to the hole 142. A condensing lens 147 is fixed in the hole 142 of the support member 141.

The controller 133 is provided with a drive mechanism (not shown) configured to move the movable member 143, and the user can move the movable member 143 by operating the remote controller 134 so that the movable member 143 is located at any one of two positions.

When the movable member 143 is located at a first position in the hole 142, the condensing lens 144 is located at a position where the condensing lens 144 emits light from the light source device 146 to the proximal end surface of the probe 131. When the movable member 143 is located at a second position in the hole 142, the prism 145 is located at a position where the prism 145 receives light from the proximal end surface of the probe 131, reflects the light by the reflecting surface of the prism 145, and emits the light to the spectroscope 7.

As described above, the controller 133 is a switching unit switchable between a first state and a second state. The controller 133 as a switching unit emits light for temperature measurement to the spectroscope 7 in the first state, and emits illumination light from the light source device 146 to the proximal end portion of the probe 131 in the second state.

A signal line 7a from the spectroscope 7 is connected to the PC 8. Reflected light of the subject from the observation window 103 provided on the distal end surface of the distal end portion 13 passes through the optical fiber bundle 101, and is incident on the image pickup unit 4.

Therefore, an effect similar to the effect of the fifth modification can also be obtained by the present sixth modification.

Note that the endoscope 2 has a bending portion 135 on the proximal end side of the distal end portion 13 in the present modification. The bending portion 135 includes plural bending pieces, and the bending portion 135 is bent in upward, downward, rightward and leftward directions of the endoscopic image by towing and slackening four bending wires inserted through an inside of the insertion portion 11.

One end of a cable 136 through which the four bending wires are inserted is connected to the adapter 21. The other end of the cable 136 is connected to a motor unit 137. The motor unit 137 is connected to the controller 138. A remote controller 139 is connected to the controller 138.

The motor unit 137 has a motor, a gear, and the like which are configured to drive the four wires so as to tow or slacken the four wires based on a control signal from the controller 138. The user operates the remote controller 139, for example, operates a joystick of the remote controller 139, whereby the controller 138 outputs a control signal to the motor unit 137. By operating the remote controller 139, the user can direct the distal end portion 13 in a direction in which the user desires to observe in the subject.

Therefore, by operating the remote controller 134, the user can measure the temperature of the subject and the like by emitting illumination light from the distal end portion of the probe 131 or leading light from the subject to the spectroscope 7.

Note that an illumination range of illumination light at the distal end portion 132 of the probe 131 may be changed.

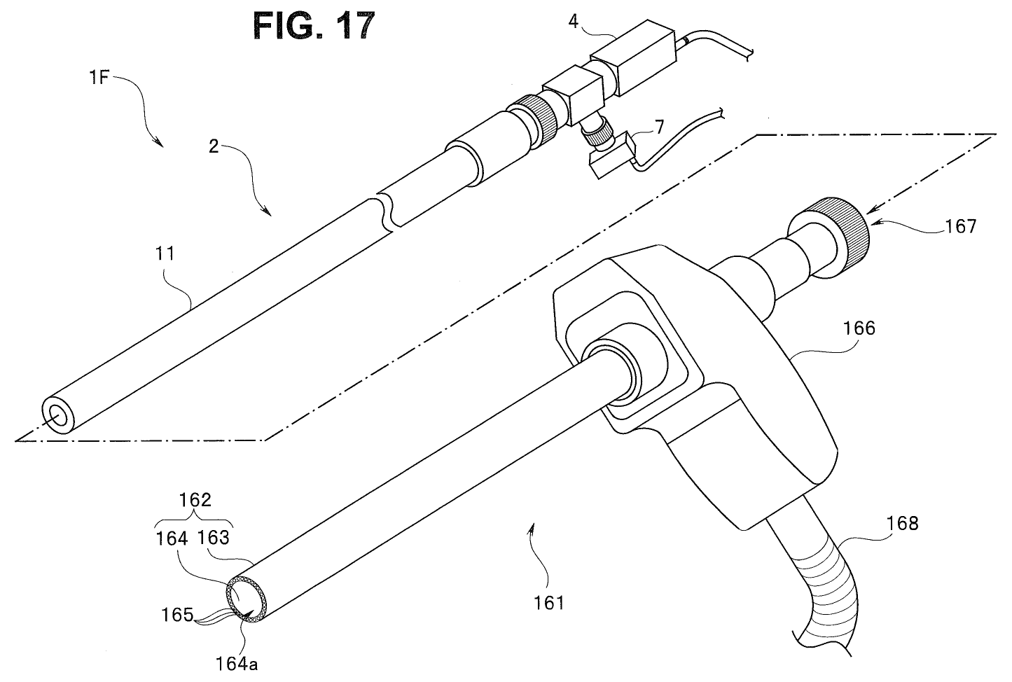

FIG. 16 is a perspective view showing a drive mechanism of the distal end portion 132 of the probe 131. A ferrule (not shown) having electrical conductivity and four piezoelectric elements 151 arranged so as to surround the ferrule are arranged at the distal end portion of the optical fiber bundle 101A of the probe 131. The proximal end portions of the four piezoelectric elements 151 are fixed to a fixing portion 152. By applying a drive voltage having a predetermined waveform to each piezoelectric element 151, each piezoelectric element 151 expands and contracts, whereby the distal end of the optical fiber bundle 101A of the probe 131 can be moved spirally within a predetermined plane, for example. The driving of the four piezoelectric elements 151 can be performed by operating the remote controller 134.

Therefore, the user can move the distal end portion of the optical fiber bundle 101A within a predetermined range by operating the remote controller 134. Accordingly, the movement of the distal end portion of the optical fiber bundle 101A makes it possible to illuminate the subject within a range W indicated by a two-dotted chain line in FIG. 16 and measure the temperature of the subject within the range W.

### (Seventh Modification)

The endoscope system according to the above-described embodiment has the lighting mechanism to obtain an endoscopic image, but the lighting mechanism may be detachable.

FIG. 17 is a diagram showing a configuration of an endoscope system having a detachable lighting unit according to a seventh modification.

The lighting unit 161 of the endoscope system 1F has a double pipe 162. The double pipe 162 has an outer pipe 163 and an inner pipe 164. The outer diameter of the inner pipe 164 is smaller than the inner diameter of the outer pipe 163. The inner pipe 164 is inserted into the outer pipe 163.

A gap is formed between the outer peripheral surface of the outer pipe 163 and the inner peripheral surface of the inner pipe 164, and a light guide including plural optical fibers 165 is inserted in parallel to the axial direction of the double pipe 162 through the gap.

A proximal end portion of the double pipe 162 is connected to a main body portion 166 of the lighting unit 161. An opening portion 167 into which the endoscope 2 is inserted is formed on an opposite side of the double pipe 162 to the main body portion 166. The opening portion 167 and a distal end opening portion 164a of the inner pipe 164 of the double pipe 162 communicate with each other, so that the insertion portion 11 of the endoscope 2 can be inserted through the opening portion 167 and the distal end opening portion 164a.

A cable 168 extends from the main body portion 166, and a light guide including plural optical fibers 165 is inserted through the cable 168. The proximal end portions of the plural optical fibers 165 receive light from a light source device (not shown).

As a result, the user attaches the lighting unit 161 to the endoscope 2 so that the distal end portion of the insertion portion 11 is positioned at the distal end opening portion 164a of the inner pipe 164 to emit illumination light from the distal end portions of the plural optical fibers 165, thereby allowing the subject to be illuminated.

Note that the lighting unit 161 has the elongated double pipe 162 and the main body portion 166 in FIG. 17, but the lighting unit 161 may be configured in a ring-like shape so as to be attached to only the distal end portion 13 of the insertion portion 11.

### (Eighth Modification)

In the temperature measurement system of the above-described embodiment, the branching unit is provided to the endoscope to supply branched light to the spectroscope. However, in the temperature measurement system of the present modification, a two-color thermometer is configured by two filters and two image pickup devices, and one of the image pickup devices is used to acquire an endoscopic image.

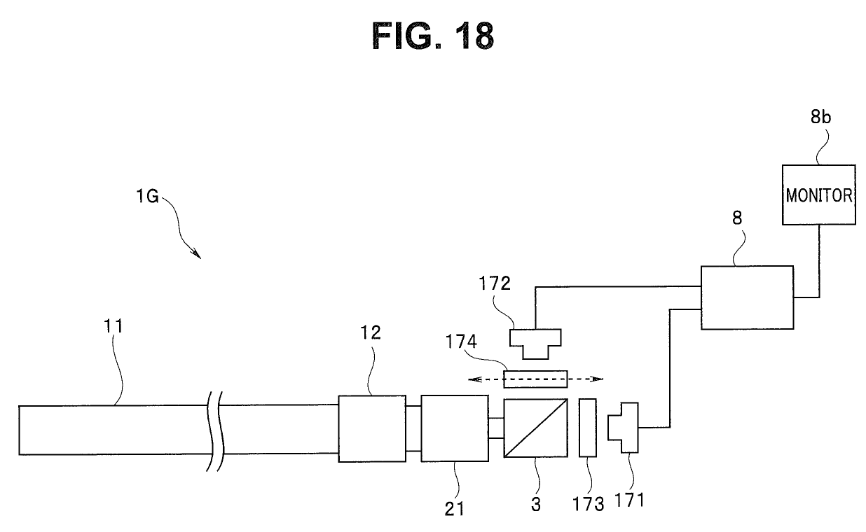

FIG. 18 is a configuration diagram of a temperature measurement system according to an eighth modification.

The temperature measurement system 1G is a two-color thermometer having two image pickup devices 171 and 172 and two filters 173 and 174. The respective image pickup devices 171 and 172 are image sensors, and arranged at a position where light reflected from the beam splitter 3 passes and a position where light transmitted through the beam splitter 3 passes. The filter 173 is a bandpass filter configured to transmit only light in one wavelength band out of two wavelengths used in the two-color thermometer. The filter 174 is a bandpass filter configured to transmit only light in the other one wavelength band out of the two wavelengths used in the two-color thermometer.

Light transmitted through the eyepiece unit 12 from the insertion portion 11 of the endoscope 2 is split into two parts by the beam splitter 3. One light from the beam splitter 3 passes through the filter 173 and is received by the image pickup device 171. The other light from the beam splitter 3 passes through the filter 174 and is received by the image pickup device 172. Each of the image pickup devices 171 and 172 has a device which is capable of receiving light of a specific wavelength to function as a two-color thermometer. In other words, the spectroscope is configured by the image pickup devices 171 and 172.

The filter 174 is arranged between the image pickup device 172 and the beam splitter 3, but is configured to be movable between a first position on an optical path from the beam splitter 3 to the image pickup device 172 and a second position deviated from the optical path.

Movement of the filter 174 may be performed manually by the user, or may be performed by electrical-driving using a drive mechanism such as a motor through a user's operation of a switch or the like.

When the filter 174 is located at the first position, the two image pickup devices 171 and 172 receive light of two wavelengths, and each of the image pickup devices 171 and 172 outputs an image signal corresponding to a spectral radiance value to the PC 8. The PC 8 calculates two spectral radiances from the two image signals, and the temperature calculating section 32 calculates the temperature of the subject from the two calculated spectral radiances, and displays the temperature of the subject on the monitor 8b.

When the filter 174 is located at the second position, the image pickup device 172 receives light from the beam splitter 3, and outputs an image signal to the PC 8. The image generating section 31 of the PC 8 generates an endoscopic image based on the image signal from the image pickup device 172, and displays the endoscopic image on the monitor 8b.

Therefore, according to the present modification, the temperature measurement system 1G generates an endoscopic image by using one of the two image pickup devices for the two-color thermometer.

### (Ninth Modification)

In the above-described sixth modification, the distal end portion of the optical fiber bundle protruding from the distal end portion of the insertion portion 11 is driven for scanning. However, in the present ninth modification, a scan operation is performed behind the proximal end portion of the optical fiber bundle.

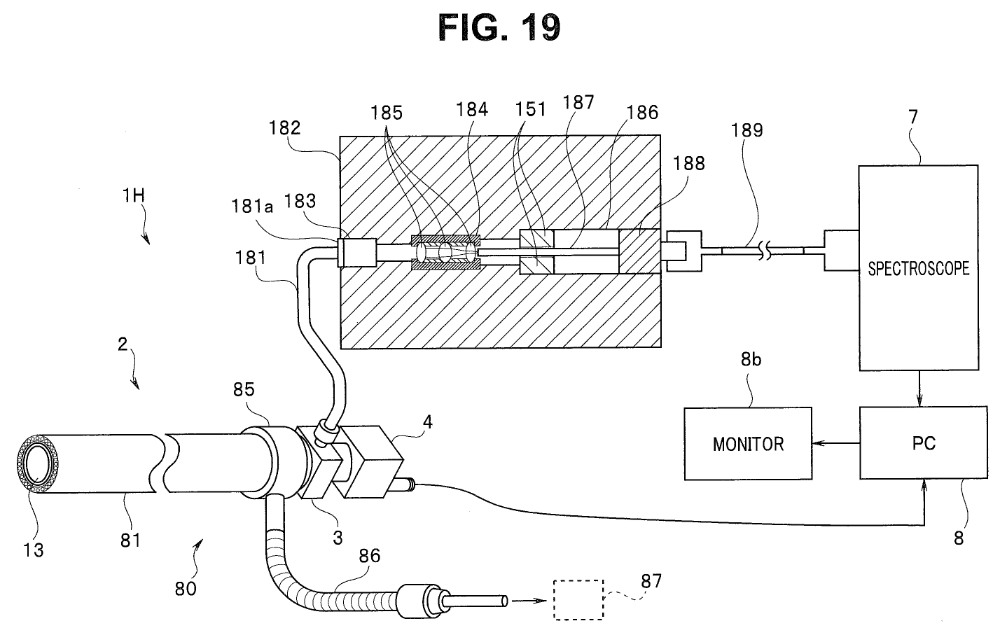

FIG. 19 is a configuration diagram of an endoscope system 1H according to the ninth modification.

The endoscope 2 is the same as the endoscope of the above-described embodiment, but is a front-view type endoscope. In other words, the endoscope 2 does not have the prism 15 at the distal end portion of the endoscope of the above-described embodiment. The image pickup unit 4 is connected to the proximal end portion of the endoscope 2 via the beam splitter 3, and a signal of the image pickup unit 4 is output to the PC 8.

The lighting unit 80 has components which are substantially similar to the components of the above-described lighting unit shown in FIG. 10. Light from the light source device 87 connected to the light guide cable 86 is emitted from the distal end surface of the double pipe 81.

Light branched from the beam splitter 3 is input to a scan unit 182 via a light guide cable 181 including plural optical fiber bundles. A connector 181a of the light guide cable 181 is connected to a connector 183 of the scan unit 182.

The scan unit 182 has a light condensing unit 184 configured to condensing light from the light guide cable 181. The light condensing unit 184 includes plural condensing lenses 185 provided in the tubular member.

The scan unit 182 further includes a fiber movable unit 186. The fiber movable unit 186 includes four piezoelectric elements 151 described with reference to FIG. 16 and a fiber portion 187 including one or plural optical fibers. A proximal end portion of the fiber portion 187 is supported by a support portion 188.

The four piezoelectric elements 151 are driven by a drive circuit (not shown) so as to move the distal end of the fiber portion 187, for example, spirally on a predetermined plane.

A proximal end of the fiber portion 187 is connected to a distal end portion(s) of an optical fiber(s) in a light guide cable 189, and light incident on the distal end of the fiber portion 187 is incident on the spectroscope 7.

According to the configuration of the present modification, light branched from the beam splitter 3 forms an image of a region corresponding to an endoscopic image in the light condensing unit 184.

The scan unit 182 scans light of an image formed in the light condensing unit 184, for example, in a spiral shape. The fiber portion 187 outputs light obtained by scanning to the spectroscope 7 via the light guide cable 189.

As a result, the temperature signal obtained in the spectroscope 7 is supplied to the PC 8, and the temperature of the object is calculated based on output signals of plural spectral radiance values from the spectroscope 7 in the PC 8.

### (Tenth Modification)

In the above-described embodiment, the temperature of the region corresponding to the endoscopic image is measured. However, in the present modification, the temperature of a predetermined region in an obtained endoscopic image is measured.

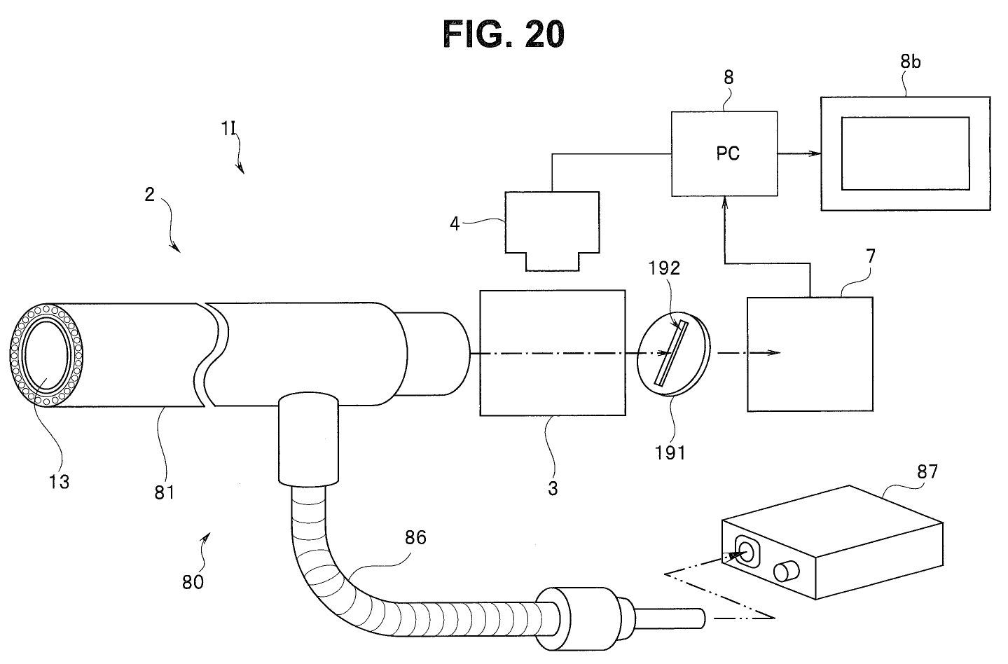

FIG. 20 is a configuration diagram of an endoscope system according to a tenth modification.

The endoscope 2 of the endoscope system 1I is the same as the endoscope of the above-described embodiment, but is a front-view type endoscope. The image pickup unit 4 is connected to the proximal end portion of the endoscope 2 via the beam splitter 3, and a signal of the image pickup unit 4 is output to the PC 8. Light branched by the beam splitter 3 is output to the image pickup unit 4.

The lighting unit 80 has components which are substantially similar to the components of the above-described lighting unit of FIG. 10. Light from the light source device 87 connected to the light guide cable 86 is emitted from the distal end surface of the double pipe 81.

Light transmitted through the beam splitter 3 is emitted to the spectroscope 7. A slit unit 191 is arranged between the beam splitter 3 and the spectroscope 7.

The slit unit 191 has a hole 192 through which only a part of light from the object passes. The spectroscope 7 receives light transmitted through the hole 192, and outputs output signals of plural spectral radiance values. In other words, the slit unit 191 is a light shielding member which is arranged between the beam splitter 3 and the spectroscope 7, and has the hole 192 which is a through slit.

Here, since the hole 192 has an elongated shape, light in a range that crosses an image of the object in a predetermined direction out of the image of the object is incident on the spectroscope 7.

The PC 8 receives the image signal from the image pickup unit 4 and the output signals of the plural spectral radiance values from the spectroscope 7, and displays the endoscopic image and the temperature information on the monitor 8b.

Therefore, in the temperature measurement system of the present modification, it is also possible to measure the temperature in a predetermined range of the object together with the endoscopic image obtained in the image pickup unit 4.

As described above, according to the above-described embodiment and each of the modifications, a temperature measurement system and an endoscope that are capable of acquiring an image of a subject and measuring the temperature of a desired site of the subject in a non-contact manner can be provided.

The present invention is not limited to the above-described embodiment, and various modifications, alterations, and the like can be made without changing the subject matter of the present invention.

What is claimed is:

1. A temperature measurement system comprising:
at least one observation optical system;
at least one branching unit configured to branch light from the observation optical system into first light including visible light from a subject and second light for temperature measurement;
at least one image pickup device configured to receive the first light from the branching unit and pick up an image of the subject;
at least one spectroscope configured to receive the second light from the branching unit and measure a plurality of spectral radiances of the subject; and
a processor configured to calculate a temperature of the subject based on an output signal including the plurality of spectral radiances of the spectroscope.

2. The temperature measurement system according to claim 1, wherein the branching unit includes a beam splitter configured to receive the light from the observation optical system and split the light into the first light and the second light, the image pickup device receives the first light from the beam splitter, and the spectroscope receives the second light from the beam splitter.

3. The temperature measurement system according to claim 1, further comprising a chromatic aberration correction optical system arranged between the observation optical system and the beam splitter and configured to correct chromatic aberration occurring in the observation optical system.

4. The temperature measurement system according to claim 3, wherein the chromatic aberration correction optical system is formed of multi-component glass.

5. The temperature measurement system according to claim 4, wherein the observation optical system comprises an optical member of quartz glass.

6. The temperature measurement system according to claim 1, further comprising an optical system for focus adjustment configured to adjust a focal position in the observation optical system, wherein the optical system for focus adjustment is provided between the branching unit and the image pickup device or between the observation optical system and the branching unit.

7. The temperature measurement system according to claim 1, further comprising an image generating section configured to generate an image of the subject from an image pickup signal from the image pickup device, wherein the temperature measurement system includes two of the observation optical systems, two of the branching units, two of the image pickup devices, and two of the spectroscopes, the processor generates a 3D image from images obtained by the two image pickup devices, and the processor calculates a temperature of the subject from a plurality of spectral radiance values measured by the two spectroscopes.

8. The temperature measurement system according to claim 1, wherein the observation optical system comprises an optical fiber bundle including a plurality of optical fibers.

9. The temperature measurement system according to claim 1, wherein the observation optical system includes an optical fiber bundle including a plurality of optical fibers, and the branching unit branches light passing through a part of the optical fiber bundle as the first light to the image pickup device, and branches light passing through another part of the optical fiber bundle as the second light to the spectroscope.

10. The temperature measurement system according to claim 1, further comprising a switching unit switchable between a first state and a second state, wherein the observation optical system includes a first optical fiber bundle including a plurality of optical fibers and a second optical fiber bundle including a plurality of optical fibers, the first optical fiber bundle irradiates the image pickup device with the first light, and the switching unit is provided on a proximal end side of the second optical fiber bundle to emit the second light to the spectroscope under the first state and emit illumination light from a light source device to a proximal end portion of the second optical fiber bundle under the second state.

11. The temperature measurement system according to claim 1, further comprising a switching unit switchable between a first state and a second state, and a probe that is connected to the switching unit at a proximal end portion of the probe and includes a plurality of optical fibers, wherein the observation optical system includes an optical fiber bundle including a plurality of optical fibers; the first optical fiber bundle irradiates the image pickup device with the first light; the switching unit emits the second light to the spectroscope under the first state and emits illumination light from a light source device to a proximal end portion of the probe under the second state; the observation optical system is arranged inside an insertion portion of an endoscope; and the probe is arranged inside a channel provided in the insertion portion.

12. The temperature measurement system according to claim 1, wherein the spectroscope comprises the image pickup device.

13. The temperature measurement system according to claim 1, further comprising a light shielding member arranged between the branching unit and the spectroscope and including a through slit.

14. An endoscope comprising:
an observation optical system;
a branching unit configured to branch light from the observation optical system into first light including visible light from a subject and second light for temperature measurement; and
an image pickup device configured to receive the first light from the branching unit and pick up an image of the subject.
